(19) United States
(12) Patent Application Publication
(10) Pub. No.: US 2026/0334771 A1
(43) Pub. Date: Oct. 29, 2026

(54) IN VIVO CLEANING DEVICE ACCOMMODATING SURGICAL SCOPES HAVING DIFFERENT IMAGING ELEMENT ANGULAR ORIENTATIONS

(71) Applicant: ClearCam, Inc., Austin, TX (US)

(72) Inventors: Tremaan Alexander Spearman-White, Austin, TX (US); David James Oh, Austin, TX (US); Alexander Ross Cohen, Austin, TX (US); Etse-Oghena Y. Campbell, Austin, TX (US); Christopher Robert Idelson, Austin, TX (US)

(73) Assignee: ClearCam, Inc., Austin, TX (US)

(21) Appl. No.: 19/190,603

(22) Filed: Apr. 26, 2025

(51) Int. Cl. A61B 1/12; A61B 1/313

(52) U.S. Cl. CPC A61B 1/126; A61B 1/3132

(57) ABSTRACT

A wiper body for an apparatus adapted to clean an imaging element of a surgical instrument comprises a first arcuate wiper edge segment and a second arcuate wiper edge segment. The first arcuate wiper edge segment has a first direction of curvature. The second arcuate wiper edge segment has a second direction of curvature segment opposite the direction of curvature of the first arcuate wiper edge segment. The second arcuate wiper edge segment extends contiguously from the first arcuate wiper edge segment to jointly define a front edge portion of the wiper body.

### FIELD OF THE DISCLOSURE

The disclosures made herein relate generally to cleaning devices for imaging elements of surgical scopes and, more particularly, to in vivo cleaning devices each accommodating a plurality surgical scopes having a different respective imaging element angular orientation.

### BACKGROUND

Surgical procedures utilizing in vivo visualization of target surgical sites are well known as a form of a concealed operation site. Examples of these surgeries include, but are not limited to, endoscopic surgery, laparoscopic surgery, thoracoscopic surgery and the like. These surgical procedures all utilize a surgical instrument having an integrated visualization device for providing in vivo visualization of a target surgical site within a surgical space of the patient. Although it is common for the surgical instrument to be referred to in the context of the specific type of surgical procedure (e.g., endoscope for endoscopic surgery, laparoscope for laparoscopic surgery, and the like), these surgical instruments are generally referred to herein as an “endoscope”.

As shown in FIG. 16, an endoscope 1 used in these surgical procedures is characterized as having a user interface portion 5 and an extension portion 10 connected at its proximate end 15 to the user interface portion 5. Scopes for endoscopic surgery generally have an extension portion that is substantially flexible, whereas scopes for other types of surgical procedures – e.g., for laparoscopic surgery, as shown in FIG. 16 – generally have an extension portion 10 that is substantially rigid. The extension portion 10 has an imaging element 20 such as a lens at its distal end portion 25. The imaging element 20 can have an exposed surface that is generally flush with or that defines an end face of the extension portion 10. However, in some endoscopes, the imaging element 20 may have an exposed surface that is recessed relative to a surrounding support structure, may have a non-planar surface (e.g., convex or concave), or otherwise not flush with a surrounding support structure. The imaging element 20 is connected to an optical fiber or other image transmitting element that is internal to the endoscope. The optical fiber or other image transmitting element extends along the length of the extension portion 10 and terminates at an eyepiece 30 on the user interface portion 5. The eyepiece 30 enables the imaging element 30 to be connected to a visualization device (e.g., a camera connected to a visual display console) through which target surgical sites may be viewed by surgery personnel.

During a surgical procedure using an endoscope, the exposed surface of the imaging element thereof may become impaired due to one or more in vivo scenarios. Examples of these scenarios include the exposed surface of the imaging element becoming fogged with moisture within the surgical space, or the exposed surface of the imaging element may be contaminated (e.g., smeared) by blood or other bodily fluids or tissues (e.g., interstitial fluid, fat tissue or the like). Traditionally, there are two primary different endoscope cleaning methods that are commonly utilized. The first of these cleaning methods is to remove the endoscope from the body, wipe the imaging element clean, and reinsert the endoscope into the body. This method, though effective, is time consuming and causes the surgeon to lose visual of the surgical site, which may be considered dangerous, as surgical instruments typically remain inside the body. This method can also subject the patient to a higher risk of infection. The second of these cleaning methods is to wipe the exposed surface of the imaging element upon a nearby organ or tissue. Although the endoscope remains inside the body, takes less time to clean and does not potentially compromise the surgical site, this method is often not sufficiently effective either due to the “cleaning” surface not providing effective cleaning performance or simply further contaminating the exposed surface of the imaging element. Also, when using either of these cleaning methods, the surgeon must undesirably spend time relocating the endoscope to the surgical site after cleaning the imaging element.

At a minimum, traditional approaches for cleaning the exposed surface of the imaging element may be a hindrance and an annoyance for surgeons and may offer poor cleaning performance. Additionally, the action of cleaning the exposed surface of the imaging element increases the length of time a surgical procedure takes, thereby decreasing the amount of operating room (OR) time available to the hospital. It is also costly for hospitals, patients, and insurance companies due to wasted time, and possibly surgical complications and post-surgical infection rates. Additionally, as patients undergo longer procedures, their time spent under anesthesia increases. Increased time under anesthesia has been shown to correlate to a rise in surgical complication rates and post-surgical infection rates. Thus, the added time associated with traditional approaches for cleaning the exposed surface of the imaging element is not only a hindrance, but also potentially medically and financially costly.

Thus, to maintain required visualization of target surgical sites, it is desirable to clean an exposed surface of an imaging element of an endoscope while the distal end portion of the endoscope remains in a concealed operation site (e.g., an endoscope in vivo). Known methods and devices that are intended to provide for cleaning of a surface of such cleaning devices when still within the concealed operation site (e.g., an in vivo endoscope cleaning device) have one or more shortcomings (e.g., lack efficacy, interfere with the surgical procedure, require significant alteration to a surgeon’s preferred surgical technique, etc.). Additionally, a recessed or non-planar exposed imaging element surface is also known to adversely impact cleaning of the imaging element. Still further, endoscopes of a given line (e.g., manufacturer’s series) of endoscopes may have different imaging element angular orientation although otherwise being identical in construction. For example, a first model of a line of endoscopes may have a first imaging element angular orientation (e.g., 0-degree) and a second model of a line of endoscopes may have a second imaging element angular orientation different than the first imaging element angular orientation (e.g., 30-degree). From at least inventorying and manufacturing standpoints, the need for two or more different in vivo endoscope cleaning device to accommodate different endoscopes that are otherwise the same aside from imaging element angular orientation.

Therefore, an in-vivo cleaning device that accommodates two or more endoscopes that are otherwise compatible therewith but each having a given different imaging element angular orientation would be advantageous, desirable and useful.

### SUMMARY OF THE DISCLOSURE

The disclosures made herein are directed to embodiments of an in-vivo cleaning device that accommodate a plurality of endoscopes (i.e., surgical scopes) that are otherwise compatible therewith but each having a respective different imaging element angular orientation (e.g., 0-degree and 30-degree). Such embodiments of the in-vivo cleaning device enable each of the plurality of endoscopes to be engaged therewith in a fixed seated position. When so engaged, a wiper assembly of the in-vivo cleaning device enables the in-vivo cleaning device to provide required cleaning of an imaging element of each of the plurality of endoscopes even in view of the different respective imaging element angular orientations thereof. Accordingly, such embodiments of the in-vivo cleaning device advantageously and beneficially eliminate burden associated with having to otherwise inventory and manufacture a plurality of in-vivo cleaning devices to accommodate the different respective imaging element angular orientations.

In one or more embodiments, an endoscope cleaning device comprises a user interface body and an elongated body. The user interface body has an endoscope engagement portion and an elongated body engagement portion. The endoscope engagement portion has engagement surfaces each matingly engageable with a respective engagement surface of a user interface portion of an endoscope for retaining the endoscope in a fixed seated position (i.e., rotationally and axially) relative to a centerline longitudinal axis of the user interface body relative to the user interface body. A first reference plane extends vertically through the centerline longitudinal axis of the user interface body. The elongated body is fixedly attached at a proximate end portion thereof to the elongated body engagement portion of the user interface body. The elongated body includes a first elongated passage extending along a length of the elongated body and a second elongated passage extending along the length of the elongated body. The first elongated passage has an extension portion of the endoscope matingly engaged therein when the endoscope is engaged with the user interface body in the fixed seated position. A longitudinal centerline axis of the first elongated passage extends parallel with a longitudinal centerline axis of the second elongated passage. A second reference plane extends through both the longitudinal centerline axis of the first elongated passage and the longitudinal centerline axis of the second elongated passage. The second reference plane is rotationally offset from the first reference plane such that the first and second planes intersect along a reference axis that may extend parallel to the centerline longitudinal axis of the user interface body.

In one or more embodiments, an endoscope cleaning device comprises a user interface body, an elongated body, a wiper body, and a coupling element. The user interface body has an endoscope engagement portion and an elongated body engagement portion. The endoscope engagement portion has engagement surfaces each matingly engageable with a respective engagement surface of a user interface portion of an endoscope for retaining the endoscope in a fixed seated position (i.e., rotationally and axially) relative to a centerline longitudinal axis of the user interface body. A first reference plane extends vertically through the centerline longitudinal axis of the user interface body. The elongated body is fixedly attached at a proximate end portion thereof to the elongated body engagement portion of the user interface body. The elongated body includes a first elongated passage extending along a length of the elongated body and a second elongated passage extending along at least a portion of the length of the elongated body. A centerline longitudinal axis of the first elongated passage extends colinearly with the centerline longitudinal axis of the user interface body. The first elongated passage has an extension portion of the endoscope matingly engaged therein when the endoscope is engaged with the user interface body in the fixed seated position. The wiper body is longitudinally spaced away from a distal end face of the elongated body. The coupling element has a proximate end portion within the user interface body, a central portion extending within the second elongated passage, and a distal end portion extending from within the second elongated passage beyond the distal end face of the elongated body along a longitudinal centerline axis extending parallel to the centerline longitudinal axis of the first elongated passage. A second reference plane extends through both the longitudinal centerline axis of the first elongated passage and the longitudinal centerline axis extending parallel to the centerline longitudinal axis of the first elongated passage. The second reference plane is rotationally offset from the first reference plane such that the distal end portion of the coupling element is laterally spaced away from the first reference plane. The wiper body is fixedly attached to the distal end portion of the coupling element whereby rotational movement of the coupling element about a longitudinal axis thereof causes a corresponding rotational movement of the wiper body.

In one or more embodiments, an endoscope cleaning device comprises a user interface body, an elongated body, a wiper body, and a coupling element. The user interface body has an endoscope engagement portion and an elongated body engagement portion. The endoscope engagement portion has engagement surfaces each matingly engageable with a respective engagement surface of a user interface portion of an endoscope for retaining the endoscope in a fixed seated position relative to a centerline longitudinal axis of the user interface body relative to the user interface body. A first reference plane extends vertically through the centerline longitudinal axis of the user interface body. The elongated body is fixedly attached at a proximate end portion thereof to the elongated body engagement portion of the user interface body. The elongated body includes a first elongated passage extending along a length of the elongated body. The first elongated passage has an extension portion of the endoscope matingly engaged therein when the endoscope is engaged with the user interface body in the fixed seated position. A second reference plane extends through both the longitudinal centerline axis of the first elongated passage and the longitudinal centerline axis of the second elongated passage. The second reference plane is rotationally offset from the first reference plane such that the first and second planes intersect along a reference axis that may extend parallel to the centerline longitudinal axis of the user interface body. The wiper body is longitudinally spaced away from a distal end face of the elongated body. The wiper body includes a first arcuate wiper edge segment having a first direction of curvature and a second arcuate wiper edge segment having a second direction of curvature segment opposite the direction of curvature of the first arcuate wiper edge segment. The second arcuate wiper edge segment extends contiguously from the first arcuate wiper edge segment to jointly define a front edge portion of the wiper body. An entire portion of the front edge portion may extend within a single plane. The coupling element has a proximate end portion within the user interface body, a central portion extending within the second elongated passage, and a distal end portion extending from within the second elongated passage beyond the distal end face of the elongated body along the longitudinal centerline axis of the second elongated passage such that the distal end portion of the coupling element is laterally spaced away from the first reference plane. The wiper body is fixedly attached to the distal end portion of the coupling element whereby rotational movement of the coupling element about a longitudinal axis thereof causes a corresponding rotational movement of the wiper body.

In one or more embodiments, the elongated body is an extruded article, the first elongated passage is a first lumen of the extruded article and the second elongated passage is a second lumen of the extruded article.

In one or more embodiments, the longitudinal centerline axis of the second elongated passage extends parallel to the vertically-extending plane (i.e., the first reference plane).

In one or more embodiments, the elongated body being fixedly attached at the proximate end portion thereof to the user interface body includes the elongated body being fixedly attached at the proximate end portion thereof to the user interface body to inhibit movement of the user interface body relative to the proximate end portion of the elongated body.

In one or more embodiments, the first elongated passage and the second elongated passage both extend fully to a distal end face of the elongated body.

In one or more embodiments, the vertically-extending plane bisects a lens of the endoscope when the endoscope is engaged with the user interface body in the fixed seated position.

In one or more embodiments, the coupling element includes a cleaning member engaging structure within the wiper body, the cleaning member engaging structure includes a first leg portion extending along a width of the wiper body in an angled (skewed) manner relative to the front edge portion of the wiper body whereby a distance between the front edge portion and the first leg portion increased along the width of the wiper body.

In one or more embodiments, an entire portion of the front edge portion extends within a single plane and the first leg portion is substantially straight.

In one or more embodiments, a front surface of the wiper body is flat and the front edge portion lies along the front surface.

In one or more embodiments, a width of the front edge portion is uniform along an entire length of the front edge portion.

In one or more embodiments, the wiper body includes a first arcuate wiper edge segment having a first direction of curvature and a second arcuate wiper edge segment having a second direction of curvature segment opposite the direction of curvature of the first arcuate wiper edge segment.

In one or more embodiments, the second arcuate wiper edge segment extends contiguously from the first arcuate wiper edge segment whereby the first arcuate wiper edge segment and the second arcuate wiper edge segment jointly define the front edge portion.

In one or more embodiments, the first arcuate wiper edge segment extends within a first plane and the second arcuate wiper edge segment extends within a second plane offset from and parallel to the first plane.

In one or more embodiments, the coupling element (i.e., wiper control wire) extends through a coupling element receiving side face of the wiper body, the second arcuate wiper edge segment extends between the control wire receiving side face and the first arcuate wiper edge segment, and the second plane is located between a rear face of the wiper body and the first plane.

In one or more embodiments, a front surface of the wiper body is flat, the first arcuate wiper edge segment lies along the front surface, the second arcuate wiper edge segment lies along the front surface, and the first arcuate wiper edge segment is offset from the second arcuate wiper edge segment whereby the front edge portion is stepped.

In one or more embodiments, the wiper body has a coupling element receiving side face, a longitudinal refence axis extends through the coupling element receiving side face, the second arcuate wiper edge segment extends between the control wire receiving side face and the first arcuate wiper edge segment, the first and second arcuate wiper edge segment each extend parallel to the longitudinal refence axis, the second plane is located between the longitudinal refence axis and the first plane, and the wiper body has a uniform cross-section profile along an entire length of the wiper body between the longitudinal refence axis and the front edge portion.

These and other objects, embodiments, advantages and/or distinctions of the present invention will become readily apparent upon further review of the following specification, associated drawings and appended claims.

### BRIEF DESCRIPTION OF THE DRAWINGS

FIG. 1 is first perspective view of a wiper body in accordance with a first embodiment of the disclosures made herein.

FIG. 2 is front view of a wiper body in FIG. 1.

FIG. 3 is second perspective view of the wiper body in FIG. 1.

FIG. 4 is an end view of the wiper body in FIG. 1

FIG. 5 is a first perspective view of a wiper body in accordance with a second embodiment of the disclosures made herein.

FIG. 6 is a front view of the wiper body in FIG. 5.

FIG. 7 is a plan view of the wiper body in FIG. 5.

FIG. 8 is a second perspective view of the wiper body in FIG. 5.

FIG. 9 is a diagrammatic view showing relative location of a front edge portion of the wiper body in FIG. 5 relative to an end face of an endoscope.

FIG. 10 is a perspective view showing a cleaning device comprising a wiper body in accordance with one or more embodiments of the disclosures made herein.

FIG. 11 is a fragmentary perspective view of the cleaning device in FIG. 10 .

FIG. 12 is an enlarged fragmentary cross-sectional view taken along the line 12-12 in FIG. 10.

FIG. 13 A is a perspective view of the wiper body of the cleaning device in FIG. 10.

FIG. 13 B is a plan view of the wiper body of the cleaning device in FIG. 10.

FIG. 13C is an enlarged fragmentary cross-sectional view taken along the line 13C-13C in FIG. 10.

FIGS. 14A-14C are end views of the cleaning device in FIG. 10 showing respective wiper body positions relative to the distal end portion thereof (no scope is shown).

FIGS. 15A-15C are frontal views of the cleaning device in FIG. 10 showing respective wiper body positions relative to the distal end face of the extension portion of the endoscope shown in FIG. 16 (wiper body deformation and/or deflection may be pictorial and not actual.)

FIG. 16 is a perspective view showing a prior art endoscope embodied herein as a laparoscope.

### DETAILED DESCRIPTION

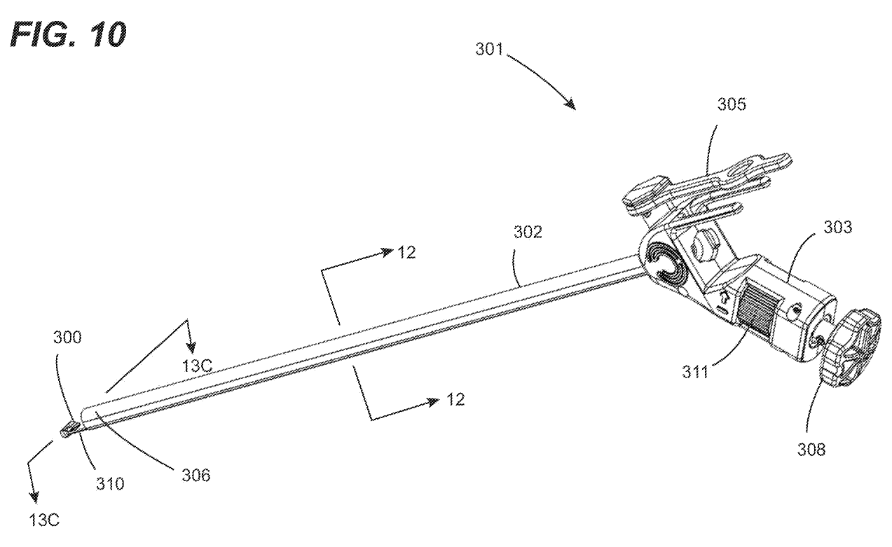

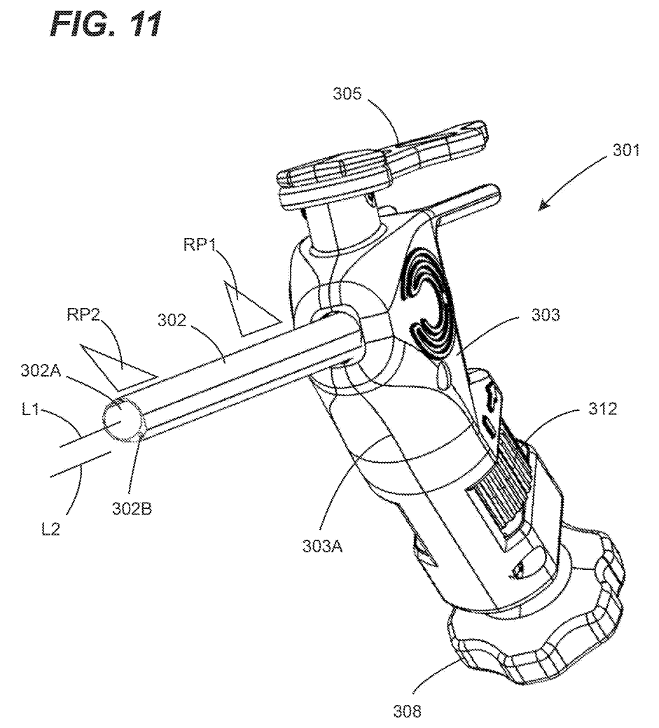

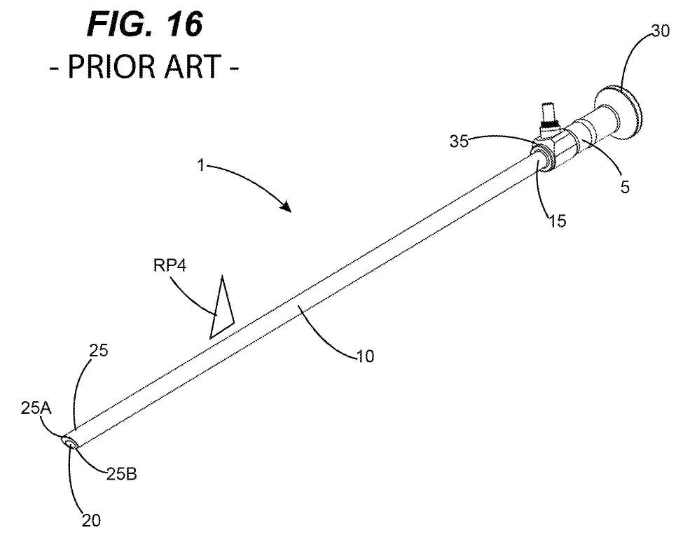

Referring now to FIGS. 10 and 11, a cleaning device 301 in accordance with one or more embodiments of the disclosures made herein is shown. US patent nos. 10932661, 10959610, 11058291, and 11013399 disclose various aspects of imaging element cleaning devices having similar construction and operability as the cleaning device 301. The cleaning device 301 is preferably, but not necessarily, configured to be used with commercially available endoscopes, such as endoscope 1 of FIG. 16. The endoscope engagement portion has engagement surfaces each matingly engageable with a respective engagement surface of a user interface portion of an endoscope for retaining the endoscope in a fixed seated position at least laterally and rotationally relative to a centerline longitudinal axis of the user interface body. A retention device may be used to retain the endoscope in a fixed seated position at least axially relative to a centerline longitudinal axis of the user interface body.

Examples of such commercially available endoscopes include, but are not limited to, endoscopes manufactured under brand names of Karl Storz, Linvatec, Olympus, Richard Wolf, Stryker and Intuitive Surgical (i.e., DaVinci). To this end, in preferred embodiments, cleaning device 301 may be engineered as endoscope-specific for a given model(s) of one or more manufacturers based on the dimensional attributes of such commercially available endoscopes. An underlying consideration of the manner in which the endoscope cleaning device 301 is engineered for an intended brand(s) or model(s) of endoscope is that there be a high level of dimensional precision between the endoscope and the cleaning device. Such dimensional precision may be characterized to include both the inhibition of any unacceptable level of relative movement between the endoscope and the cleaning device 100 and relative placement of key structural elements of the endoscope relative to those of the cleaning device 100.

As is well known in the art, engagement of the endoscope 1 with the cleaning device 300 is implemented in a manner whereby the endoscope 1 is in axially and rotationally fixed positions with respect to the cleaning device 301. Such fixed positioned may be achieved by engagement between mating surfaces of the endoscope 1 with the cleaning device 301. The objective of such mating engagement is to inhibit rotational and axial movement between the endoscope 1 and the cleaning device 301. A suitable means of retention (e.g., retention strap 305) may be used for securing the endoscope 1 in fixed engagement with the cleaning device 301.

Still referring to FIGS. 10 and 11, the cleaning device 301 has an elongated body 302 that is adapted to have theextension portion 10 of the endoscope 1 inserted. The elongated body 302 is fixedly attached at its proximate end portion to a user interface body 303 of the cleaning device 301. The elongated body 302 includes a first elongated passage 302A extending along a length of the elongated body 302 and a second elongated passage 302B extending along a length of the elongated body 302. As shown, a longitudinal centerline axis L1 of the first elongated passage 302A preferably extends parallel with a longitudinal centerline axis L2 of the second elongated passage 302B. However, in some embodiments, all or a portion of the longitudinal centerline axis L1 of the first elongated passage 302A may extend non-parallel with a longitudinal centerline axis L2 of the second elongated passage 302B. The first elongated passage 302A is configured for having the extension portion 10 of the endoscope 1 matingly engaged therein when the endoscope 1 is engaged with the user interface body 303 in the fixed seated position. The first elongated passage 302A and the extension portion 10 of the endoscope 1 are preferably jointly configured to limit lateral movement of the extension portion 10 relative to the elongated body 302.

A dimensionally predictable surface or feature of the endoscope 1 such as that of the user interface portion 5 (e.g., a handle and/or optic interface portion) abuts a mating dimensionally predictable surface or feature of the cleaning device 301 (i.e., endoscope engagement portion) when the endoscope 1 is in its fixed seated position relative to the user interface body 303. This mating surface or feature of the cleaning device 301 – such as a surface or feature of the user interface body 303 – serves as a reference structure of the cleaning device 301. With the endoscope 1 in this fully seated position on the cleaning device 301 with respect to the reference structure, a distal end portion 25 of the endoscope protrude from within an opening in the distal end portion 306 of the elongated body 302 by a known, predictable amount. Through such an interfacial arrangement and dimensional tolerances, a high level of dimensional precision between the endoscope 1 and the cleaning device 301 may be achieved. The details of such a fixed seated position are discussed below and in the US patent nos. 10932661, 10959610, 11058291, and 11013399 which are incorporated herein in their entirety by reference.

The cleaning device 301 includes a wiper body 300 in accordance with one or more embodiments of the disclosures made herein (e.g., the wiper body 200). The wiper body 300 may be fixedly coupled to a first control body 308 of the user interface body 303 via a coupling element 310 such as a wire. Rotation of the first control body 308 causes a corresponding rotational movement of the wiper body 300 for enabling cleaning of an adjacent imaging element of an endoscope engaged with the cleaning device 301.

The cleaning device 301 may include a second control body 311 that is coupled directly or indirectly to the first control body 308 and to the coupling element 310. The second control body 311 is a user interface component of a control mechanism that provides for selectively altering an axial distance between the cleaning member 300 (e.g., a reference portion thereof such as the front edge portion) and the distal end portion 306 (e.g., the distal end face) of the elongated body 302. Through rotation of the second control body 311 in a given direction, a corresponding change in the axial distance (i.e., increase or decrease) between the cleaning member 300 and the distal end portion 306 of the elongated body 302 occurs (e.g., clock-wise rotation provides lesser distance and counter clock-wise rotation provides greater distance or vice-versa). Thus, when the endoscope 1 is engaged in the fixed seated position with the cleaning device 301, the second control body 311 may be used to provide for selectively altering the axial distance between the cleaning member 300 and the exposed surface of the imaging element 20 of the endoscope 1.

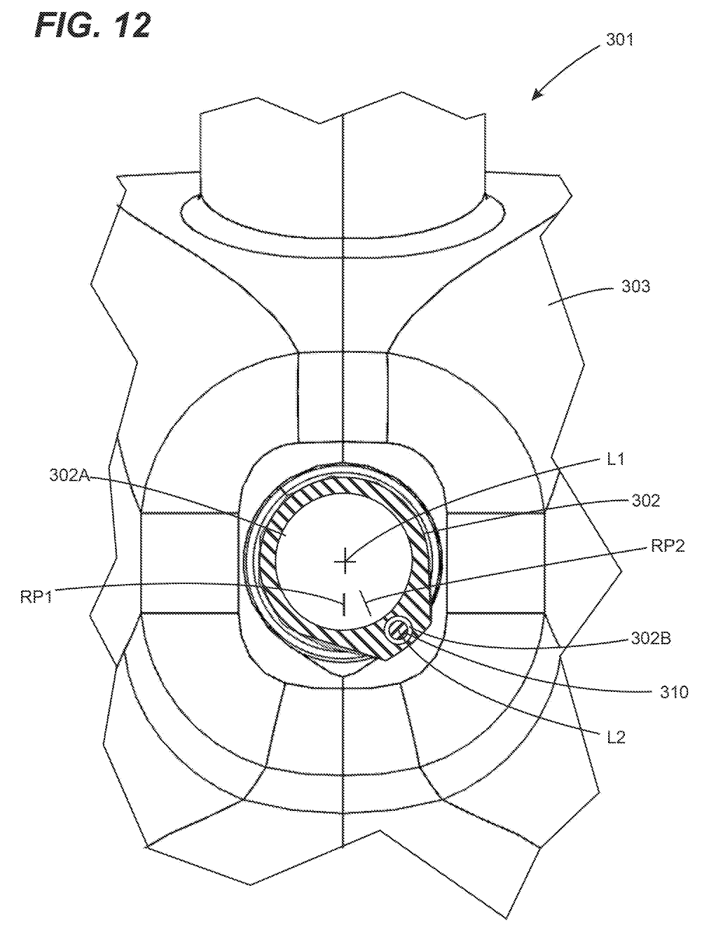

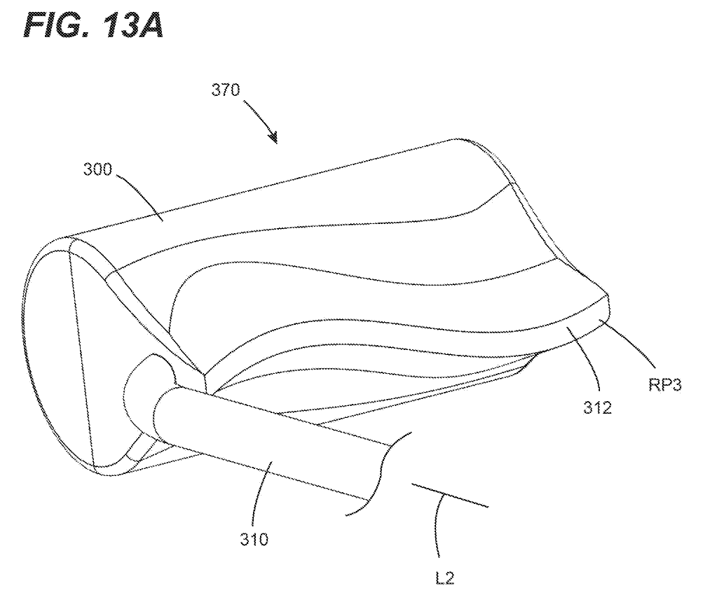

As best shown in FIGS. 11 and 12, a first reference plane RP1 extends vertically through the centerline longitudinal axis L1 of the first elongated passage 302A and a second reference plane RP2 extends through both the longitudinal centerline axis L1 of the first elongated passage 302A and the longitudinal centerline axis L2 of the second elongated passage 302B. The elongated body 302 is rotationally positioned (i.e., “clocked”) relative to the user interface body 303 such that the second elongated passage 302B is laterally spaced away from the first reference plane RP1. As shown, the elongated body 302 is rotationally positioned in a clockwise manner as viewed along the longitudinal centerline axis L2 of the second elongated passage 302B from the user interface body 303. In preferred embodiments, the elongated body 302 is rotationally offset by about 40-degrees with a tolerable of about +/- 10-degrees. In some embodiments, the elongated body 302 is rotationally offset by about greater than 15-degrees or less than 50-degrees. In this rotational configuration, the second reference plane RP2 is rotationally offset from the first reference plane RP1 such that the first and second planes RP1, RP2 intersect along a reference axis (i.e., planar intersection axis) that may extend parallel to the centerline longitudinal axis of the user interface body. The planar intersection axis is at or adjacent to the longitudinal centerline axis L1 of the first elongated passage 302A.

The elongated body 302 may be an extruded article such that the first elongated passage 302A is a first lumen of the extruded article and the second elongated passage 302B is a second lumen of the extruded article. In some embodiments, the first elongated passage 302A and the second elongated passage 302B both extend fully to an end face of the elongated body 302 at its distal end portion 306. The longitudinal centerline axis L2 of the second elongated passage 302B preferably extends parallel to the first reference plane RP1. Relative to the user interface body 303, the first reference plane RP1 is a vertically-extending plane – e.g., a plane that bisects the user interface body 303 along body separation line 303A. In preferred embodiments, the first reference plane RP1 bisects (exactly or approximately) a lens of the endoscope when the endoscope is engaged with the user interface body 303 in the fixed seated position.

Preferably, the elongated body 302 is fixedly attached at its proximate end portion to the user interface body 303 such that movement of the user interface body 303 relative to the proximate end portion of the elongated body 302 is inhibited. In some embodiments, the rotational orientation of the elongated body 302 relative to the user interface body 303 (i.e., clocking) may be selectively adjusted such as by a mechanical interfacing mechanism that allows for discrete and/or continuous positioning adjustment in the rotational position of the adjustment of the elongated body 302 relative to the user interface body 303. For example, the elongated body 302 may be manually movable between a plurality of discrete rotational position relative to the user interface body 303 or may be manually movable continuously over a range of available rotational movement and then fixedly secured in a desired rotational position thereby enabling end user adjustment and/or manufacturer adjustment.

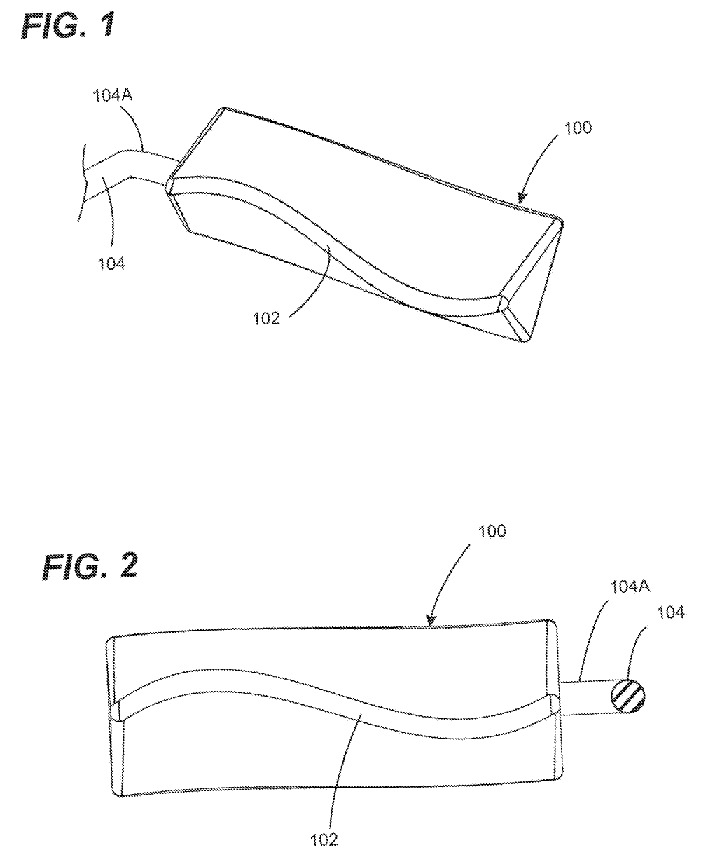

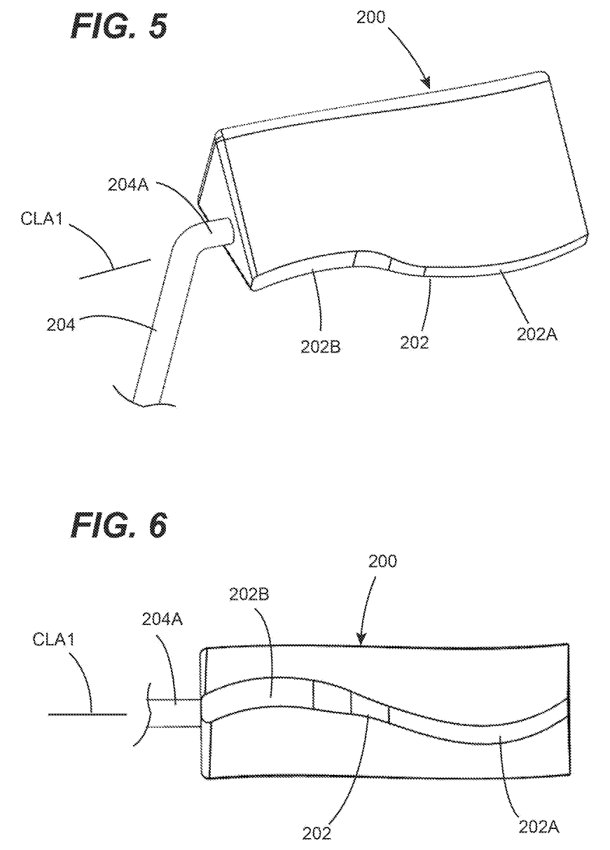

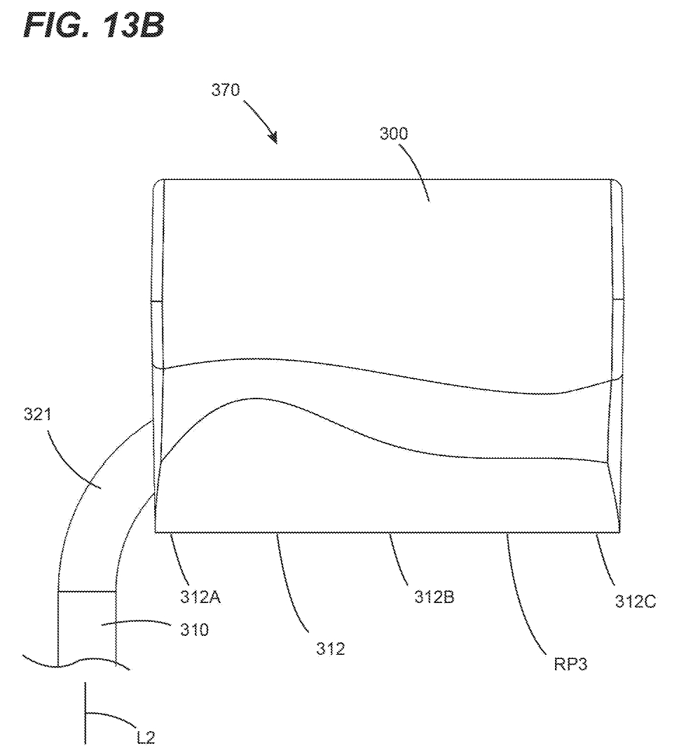

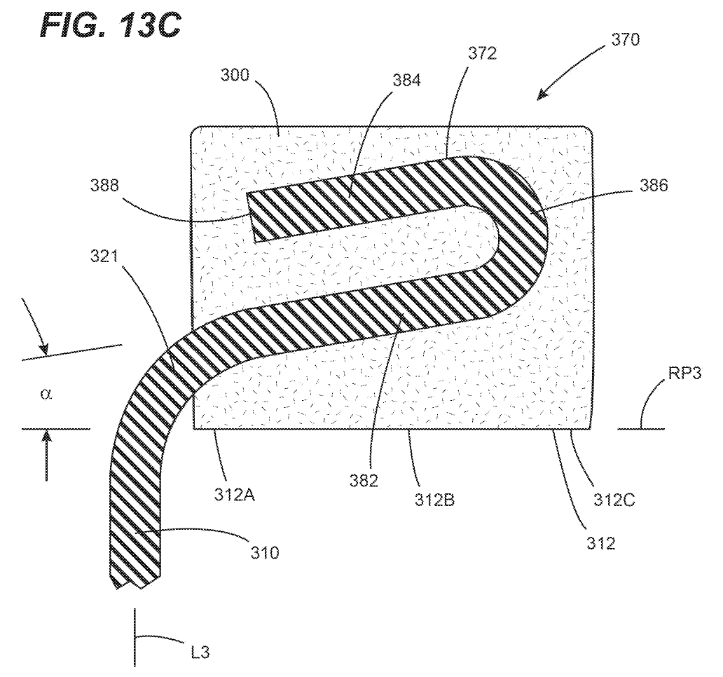

Referring now to FIGS. 13A-13C, the wiper body 300 and the coupling element 310 jointly define a wiper assembly 370 that is coupled to the first control body 308 (FIG. 10). The wiper body 300 has a front edge portion 312 that extends entirely (exactly or approximately) within a third reference plane RP3 and that has a dual curvature (e.g., S-shaped). Thus, the wiper body 300 may be of the same or similar configuration as the wiper body 100 discussed below in reference to FIGS. 1-4. However, it is disclosed herein that cleaning devices in accordance with embodiments of the disclosure made herein may utilize a different wiper body configuration – e.g., the wiper body 200 discussed below in reference to FIGS. 5-9 or a wiper body as disclosed in the prior art (e.g., US patent nos. 10932661, 10959610, 11058291, and 11013399). The third reference plane RP3 extends perpendicular to the longitudinal centerline axis L2 of the second elongated passage 302B.

As shown in FIG. 13C, the coupling element 310 includes a cleaning member engaging structure 372 at its distal end portion 321. The cleaning member engaging structure 372 is within the cleaning member 300 in fixed position whereby movement of the coupling element 310 causes a corresponding movement of the cleaning member 300 that enables cleaning of the imaging element 20 of the endoscope 1. In some embodiments, the cleaning member 300 may be molded or cast onto the cleaning member engaging structure 372. In some embodiments, the coupling element 310 may be made from a metallic material such as, for example, stainless steel (e.g., 316 or 316 LVM stainless steel). In some embodiments, the cleaning member 300 may be made from a polymeric material having elastomeric rubber, elastomeric-like or rubber-like properties (e.g., a two-part silicone rubber having a durometer of about 60 Type A, a tensile strength of about 1300 psi, an elongation of about 525%, and a tear strength of about 250 ppi).

The cleaning member engaging structure 372 preferably has a U-shaped portion defining a first leg portion 382, a second leg portion 384, and an arcuate segment 386 extending therebetween. In this respect, the cleaning member engaging structure 372 includes a plurality of segments that are spaced apart from each other. The second leg portion 384 defines a distal end face 388 of the coupling element 310 located within the cleaning member 300. The first leg portion 382 and the second leg portion 384 may be straight members that extend generally parallel to each other. As disclosed herein, the first leg portion 382 extends in an angled orientation (i.e., skewed at angle α) with respect to the third reference plane RP3 and is the closest portion of the cleaning member engaging structure 372 to the front edge portion 312.

Advantageously and beneficially, the angled orientation of the first leg portion 382 relative to the front edge portion 312 results in the cleaning member engaging structure 372 influencing deformation and/or deflection characteristics of the front edge portion 312. Because of the angled orientation of the first leg portion 382 relative to the front edge portion 312, the amount of wiper body material between the first leg portion 382 and the front edge portion 312 increases along the length of the front edge portion 312 from the end engaged by the coupling element 310. Accordingly, for a given configuration of the wiper body 300, the angled orientation of the first leg portion 382 relative to the front edge portion 312 may be used to influence deformation and/or deflection characteristics of the front edge portion 312. For example, for a given configuration of the wiper body 300 made from a single formulation of material, the angled orientation of the first leg portion 382 relative to the front edge portion 312 results in a lesser degree of deformation and/or deflection at an inner portion 312A, a greater degree of deformation and/or deflection at a middle portion 312B, and an even greater degree of deformation and/or deflection at an outer portion 312C. In these regards, through design of the wiper body, selection of the wiper body material, placement of the first leg portion 382 relative to the front edge portion 312, and placement of the first leg portion 382 relative to the front edge portion 312, the deformation and/or deflection characteristics of the front edge portion 312 (i.e., deformation and/or deflection defining aspects of the front edge portion 312) may be selectively tuned. Thus, such wiper assembly configuration provides a resulting synergistic functionality that leverages the bulk material and dimensional properties of the wiper assembly to enable a superior degree and reliability of cleaning efficacy provided by endoscope imaging element cleaning devices in accordance with embodiments of the disclosures made herein.

In totality, the inventors have discovered that the following device design elements synergistically (“synergistic device design elements”) enable the cleaning device 301 to provide utility with endoscopes having different imaging element: 1.) the aforementioned rotational offsetting (i.e., clocking) of the elongated body 302 relative to the user interface body 303; 2.) the front edge portion 312 of the wiper body 300 extending perpendicular to the longitudinal centerline axis L2 of the second elongated passage 302B; and 3.) the positional and angular orientation of the first leg portion 382 of the cleaning member engaging structure 372 relative to the front edge portion 312. For example, such synergistic device design elements enable a single cleaning device in accordance with embodiments of the disclosures made herein to provide required imaging element cleaning performance for a plurality of compatible endoscopes. Such compatible endoscopes may have the same overall design but with different imaging element angular orientations.

In an exemplary case, a first endoscope of defining an overall design may have a 0-degree (i.e., normal) imaging element orientation (or effectively but not exactly 0-degree imaging element orientation that enables an amount of preloading of the wiper body against the imaging element) where the planar imaging element face of the imaging element extends perpendicular to the longitudinal centerline axis L1 of the first elongated passage 302A) and a second endoscope of the same overall design (e.g., endoscope 1 in FIG. 16) may have a 30-degree imaging element orientation (i.e., planar imaging element face extending at a 30-degree angle relative to the longitudinal centerline axis L1 of the first elongated passage 302A). For the 0-degree (i.e., normal) imaging element orientation, the front edge portion 312 travels approximately or entirely in a plane (i.e., the third reference plane RP3) that is parallel (including being co-planar) with or near-parallel with the face of the imaging element and, thus, perpendicular or near-perpendicular to the longitudinal centerline axis L1 of the first elongated passage 302A. Accordingly, the front edge portion 312 engages the imaging element having the 0-degree imaging element orientation with uniform engagement in view of a front edge portion of a wiper body extending in a parallel or near-parallel manner with a surface (particularly, a flat surface) to be cleaned.

For the 30-degree (i.e., angled) imaging element orientation, the aforementioned synergistic device design elements advantageously and beneficially enable required cleaning performance. Notably, the imaging element 20 of the endoscope 1 having a 30-degree imaging element orientation, as shown in FIG. 16, has an oval shape as does the distal end face of the extension portion 10. Moreover, the endoscope 1 is vertically bisected by a fourth reference plane P4 (FIG. 16) whereby the fourth reference plane P4 extends through upper and lower end points 25A and 25B of the oval end face of the extension portion 10 and, thus, the oval end face of the imaging element 20 exposed at the distal end face of the extension portion 10. Accordingly, when the endoscope 1 is engaged with the cleaning device 301 in the fixed seated position, the endoscope 1 is vertically bisected (exactly or approximately) by the first reference plane RP1 that extends vertically through the centerline longitudinal axis L1 of the first elongated passage 302A.

Without being bound by any limiting wiper/coupling element deformation/deflection theory, the cleaning performance for the 30-degree imaging element orientation may enabled by the aforementioned synergistic device design elements permitting sufficient deformation and/or deflection of the wiper assembly 37 when engaged with the distal end portion 25 of the endoscope 1. Because the front edge portion 312 travels in a plane that extends perpendicular (or near perpendicular) to the longitudinal centerline axis L1 of the first elongated passage 302A, deformation and/or deflection of the wiper body 300 enables the front edge portion 312 to deform and/or deflect as the wiper body 300 is engaged with and rotated across the imaging element 20. Similarly, resiliency of the coupling element 310 enables the coupling element 310 to resiliently deflect (e.g., bend) as the wiper body 300 is engaged with and rotated across the imaging element 20.

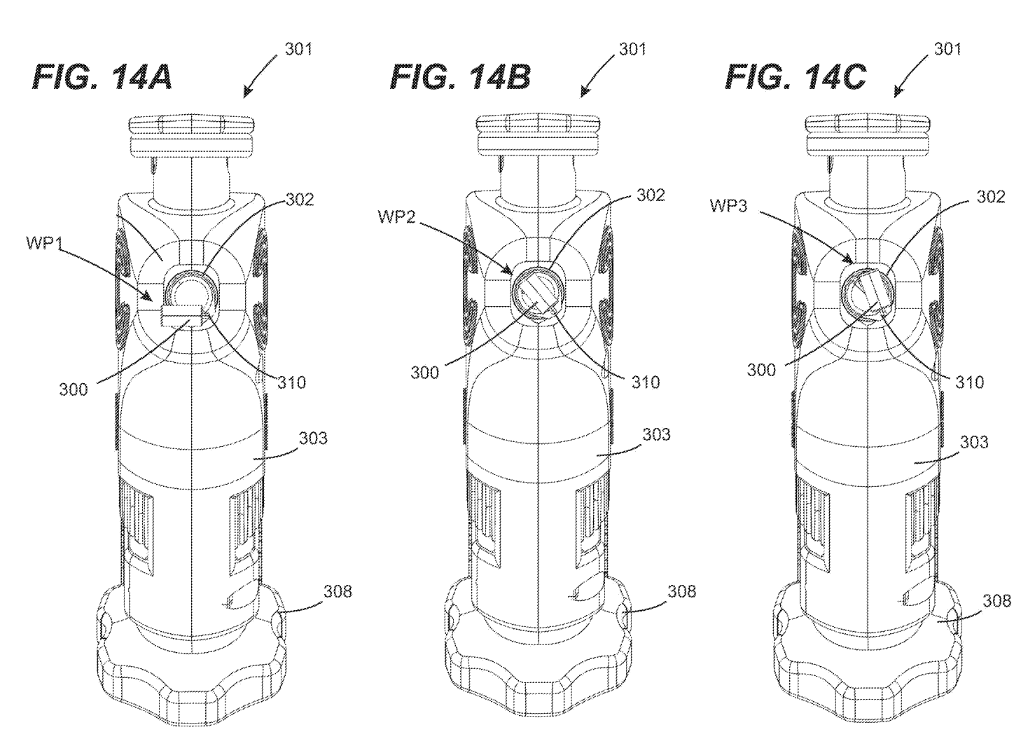

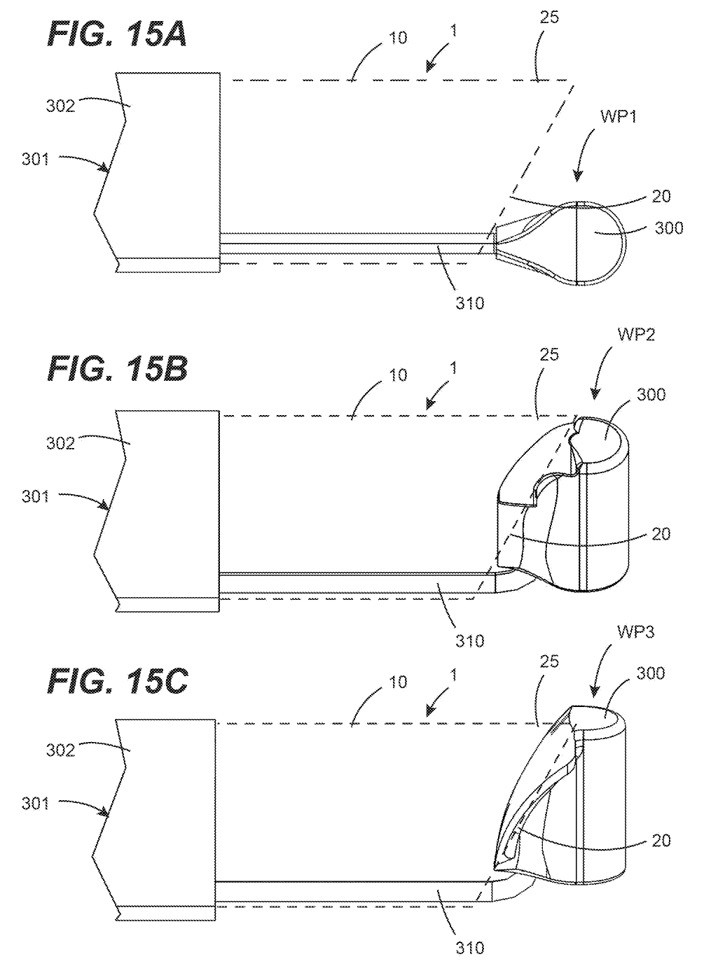

Referring now to FIGS. 14A-14C, and 15A-15C and still without being bound by any limiting wiper/coupling element deformation/deflection theory, in response to rotation of the wiper body 300 about the longitudinal centerline axis L2 of the second elongated passage 302B in the counter-clockwise direction as viewed from the user interface body 303 from an initial wiper position WP1 where the wiper body is not in contact with the endoscope 1 (FIGS. 14A and 15A), the outer portion 312C and middle portion 312B of the front edge portion 312 move into engagement with the imaging element 20 of the endoscope 1 to a partially engaged wiper position WP2 (FIGS. 14B and 15B). With further rotation of the wiper body 300 about the longitudinal centerline axis L2 from the partially engaged wiper position WP2 (FIGS. 14B and 15B), the inner portion 312A of the front edge portion 312 moves into engagement with the imaging element 20 of the endoscope 1 (FIGS. 14B and 15B) along with the outer portion 312C and middle portion 312B of the front edge portion 312. Further rotation of the wiper body 300 about the longitudinal centerline axis L2 of the second elongated passage 302B results in the wiper body fully wiping across the remaining portion of the imaging element 20 and then moving out of engagement with the endoscope 1.

Counter-clockwise rotation of wiper body 312 is implemented as a result of the elongated body 302 being rotationally positioned in a clockwise manner as viewed along the longitudinal centerline axis L2 of the second elongated passage 302B from the user interface body 303. Clockwise rotation of wiper body 312 would result in binding of the wiper body against the side face of the extension portion 10 at its distal end portion 25. However, the elongated body 302 being rotationally positioned in a counter-clockwise manner as viewed along the longitudinal centerline axis L2 of the second elongated passage 302B from the user interface body 303 would corresponding require rotation of the wiper body being in the clockwise direction as viewed from the user interface body 303.

It is disclosed herein that the first and second endoscopes may both have an angled imaging element orientation (e.g., 15-degrees and 45-degrees, 10-degrees and 30-degrees, 10-degrees and 45-degrees, and the like). It is disclosed herein that when such endoscopes both having an imaging element orientation that is at a non-normal angle to the longitudinal centerline axis L1 of the first elongated passage 302A (e.g., both angles greater than 0 degrees), the front edge portion 312 of the cleaning body 300 may similarly be at a non-normal angle relative to the second elongated passage 302B. For example, where first and second endoscopes respectively have an angled imaging element orientation of 15-degrees and 45-degrees, the front edge portion 312 of the cleaning body 300 may be at or near a 15-degree angle relative to the longitudinal centerline axis L2 of the second elongated passage 302B.

The inventors have observed less than desirable efficacy in cleaning various scope imaging element geometries of imaging element cleaning devices that utilize a traditional imaging element wiper (“wiper”). US patent nos. 10932661, 10959610, 11058291, and 11013399 disclose various aspects of imaging element cleaning devices that utilize a traditional imaging element wiper to clean the imaging element of a visualization device such as an endoscope (i.e., wiper-based imaging element cleaning devices) and are incorporated herein in their entireties by reference. These imaging element geometries include those that are “recessed,” “non-planar,” or otherwise not flush with a surrounding support structure with respect to the wiper sweeping profile. In some specific applications, a cleaning device may be configured for cleaning an imaging element (e.g., a lens) having a 30-degree taper relative to being perpendicular to a centerline longitudinal axis of the scope (as opposed to a 0-degree imaging element orientation). Such taper has been found to further complication imaging element cleaning. In these specific applications, a wiper of the cleaning device for a 30-degree taper imaging element travels in a cone-shaped pathway in 3D space relative to its fixed point of rotation when attempting to clean a 30-degree taper imaging element. The endoscope 1 discussed herein in reference to FIG. 16 is an example of an endoscope with a 30-degree taper imaging element.

Through experimentation, the inventors have found that that there is a lack of optimal contact on an imaging element by a wiper from its fixed point of rotation, regarding application to/on imaging elements having recessed or non-planar geometries with respect to the wiper actuation path. For recessed imaging elements, getting the wiper to even contact the imaging element may prove difficult. This is believed to be due to a cone-shaped actuation/sweep path attempting to contact a flat-plane imaging element. In this regard, the sweep profile is not a clean alignment, whereby imaging element contact is sometimes varied/non-optimal for cleaning.

For 30-degree taper imaging elements, larger scopes (i.e., 10mm diameter extension portion or wand) have proven more difficult to clean with a wiper-based imaging element cleaning device due to (a) less contact area for a wiper of similar scale from the 5mm diameter version (b) seemingly due to a different aspect ratio of [imaging element surface area] vs. [wiper surface area + pressure exerted on imaging element] vs. debris type and total amount of debris and effect on total surface tension of amalgamated debris on imaging element]. That is, when scaling up from wipers for scopes with a 5mm diameter extension portions that have shown relatively strong success in cleaning debris on 0-degree and 30-degree taper imaging elements, the scaled 10mm wipers in combination with 10mm imaging elements and debris types/amounts have an interaction that yields sub-satisfactory results. These sub-satisfactory results are assumed to be due to an inability to (a) contact as much imaging element surface area, apply appropriate pressure(s) across total surface area onto the imaging element from wiper, and applying enough pressure to significantly disrupt surface tension of debris on the imaging element by the wiper that enables successful and reliable imaging element cleaning performance.

For 0-degree and 30-degree taper imaging elements, it appears that liquid droplets in particular pose significant difficulty in cleaning on recessed 30-degree taper imaging elements, and this is possibly due to the liquid being pushed by the wiper into a large compiled pool of liquid with a surface tension that a standard 5mm wiper scaled to 10mm size could not overcome in a reliable manner.

The inventors have discovered wiper front edge portion configurations that provide enhanced imaging element cleaning performance for the aforementioned adverse cleaning considerations. Broadly, these front edge portion configurations are embodied in the form of an “S-shaped wiper,” as shown in FIGS. 1-8. These performance enhancing wipers may be used in imaging element cleaning devices such as, for example, those of disclosed and/or referenced herein.

Referring to FIGS. 1-4, an S-shaped wiper in accordance with a first embodiment of the disclosures made herein is shown (i.e., wiper body 100). The wiper body 100 has a non-stepped front edge portion (i.e., the cleaning edge). In view of being non-stepped, the wiper body 100 has a front edge portion 102 that extends entirely within a first front edge portion plane P1 that extends parallel to a centerline axis CLA1 of a wiper mounting portion 104A of a coupling element (e.g., a wiper control wire) 104. The wiper mounting portion 104A of the coupling element 104 is disposed within the wiper body 100. As best shown in FIG. 2, a width of the front edge portion 102 is preferably uniform along its length. In this regard, the wiper body 100 may have a generally uniform cross sectional shape along its length, width, and depth, as shown . However, the wiper body 100 may have a shape allowing for a width of the front edge portion 102 to be non-uniform along its length. In preferred embodiments, as best shown in FIG. 4, the front edge portion 102 is a flat edge as opposed to having a rounded (i.e., radiused) profile.

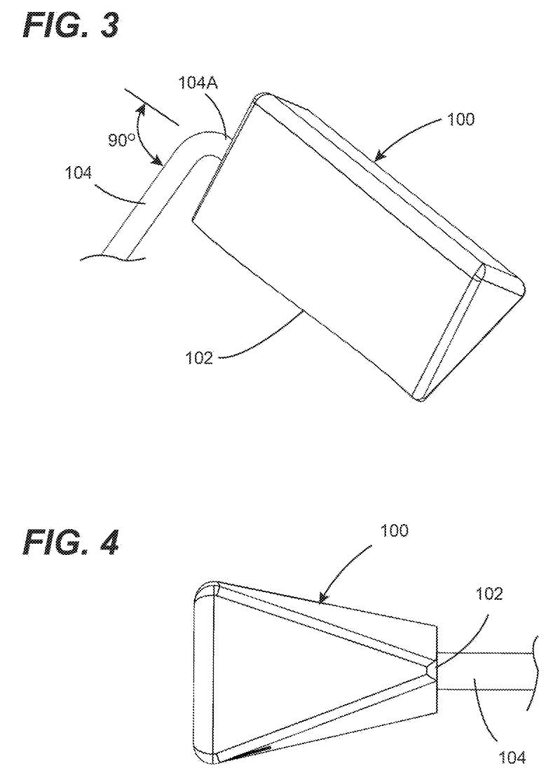

As best shown in FIG. 3, the coupling element 104 is configured for a 0-degree taper imaging element (i.e., has approximately a 90-degree bend at the wiper mounting portion 104A). However, it is disclosed herein that the wiper body 100 may be used with imaging elements of tapers other than 0-degree. In use, an imaging element cleaning device incorporating the wiper body 100 will be adapted to enable generally uniform contact force between the wiper body 100 and an imaging element of a visualization device (e.g., an endoscope) engaged with the imaging element cleaning device along the length of the wiper body 100.

Referring to FIGS. 5-8, a stepped and S-shaped wiper in accordance with a second embodiment of the disclosures made herein is shown (i.e., wiper body 200). The wiper body 200 has a stepped and S-shaped front edge portion 202. As shown, the stepped and S-shaped front edge portion 202 extends in two planes. In these regards, in view of being stepped and S-shaped, the front edge portion 202 has a first (i.e., upper) portion 202A that extends within a first front edge portion plane P1 that extends parallel to a centerline axis CLA1 of the wiper mounting portion 204A of the coupling element 204 and a second (i.e., lower) portion 202B that extends within a second front edge portion plane P2 that extends parallel to the centerline axis CLA1 of the wiper mounting portion 204A of the coupling element 204. The wiper mounting portion 204A of the coupling element 204 is disposed within the wiper body 200. The first and second front edge portion plane P1, P2 are offset from each other by a distance D. In preferred embodiments, as best shown in FIGS. 5 and 8, the front edge portion 202 is a flat edge as opposed to having a rounds (i.e., radiused) profile.

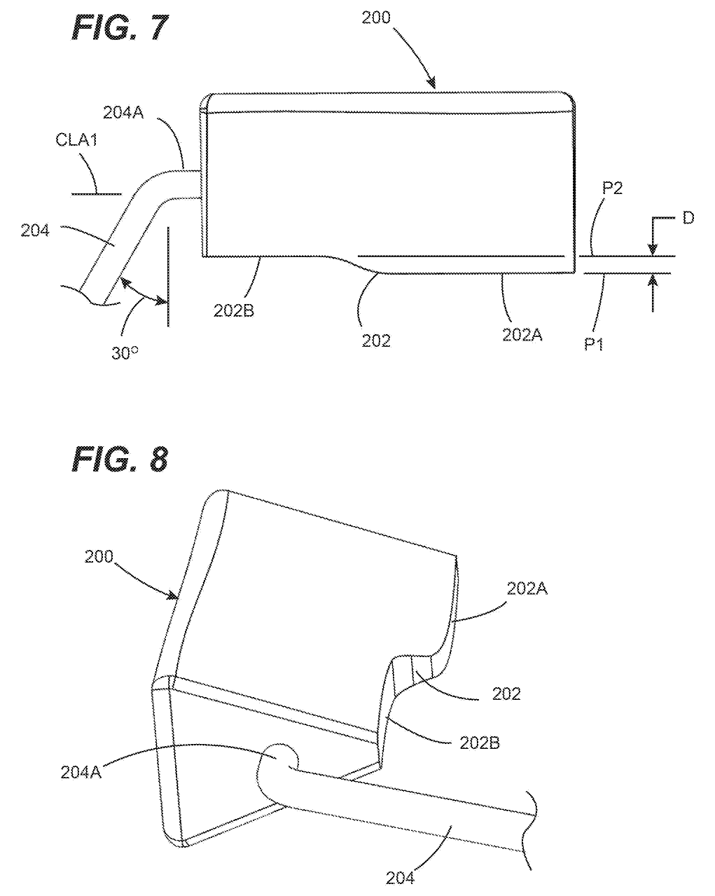

As best shown in FIG. 7, the coupling element 204 is configured for a 30-degree taper imaging element (i.e., has approximately a 120-degree bend at the wiper mounting portion 204A). However, it is disclosed herein that the wiper body 200 may be used with imaging elements of tapers other than 30-degree. As best shown in FIG. 6, a width of the cleaning edge 202 may be non-uniform along its length in view of the wiper body 100 having a generally uniform cross sectional shape along its length, width, and depth, as shown. However, the wiper body 200 may have a shape allowing for a width of the cleaning edge 102 to be generally uniform along its length.

Upon initial setting of an imaging element cleaning device incorporating the wiper body 200 (e.g., an imaging element cleaning device discussed in the one or more of the patents incorporated herein by reference), the wiper body 200 may be aligned in the middle of an imaging element of a visualization device engaged with the imaging element cleaning device. The imaging element cleaning device is preferably adjusted to a wiper body pre-load set point that compresses the “upper” portion 202A of the front edge portion 202. Because the wiper body is made of a deformable material, the wipe body compresses until the user may stop the set point at a stage where the “upper” portion 202A front edge portion 202 is deformed against the imaging element to a point where the “lower” portion 202B of the front edge portion 202 is in low-force contact with the imaging element. This preload arrangement enables the utilization of the normal force of the imaging element on the front edge portion 202 to fully engage contact of the front edge portion 202 with the engaged surface of the imaging element. In this preload arrangement, the “upper” portion 202A is highly deformed and the “lower” portion 202B is slightly deformed. However, an entire length of the front edge portion 202 is in contact with the imaging element.

In use, the wiper body 200 sweeps across the imaging element in a single direction. The wiper body 200 may be biased to work in specific direction/mode of actuation in view of its geometric profile. It is believed that the portion of the wiper body 200 that defines the front edge portion 202 is able to adjust/compensate to the cone-shaped pathway to maintain engagement between the front edge portion 202 with the imaging element even when the imaging element is recessed. Maintaining engagement between the front edge portion 202A and imaging element in this manner provides for sustained and robust cleaning performance over a substantial area of the imaging element.

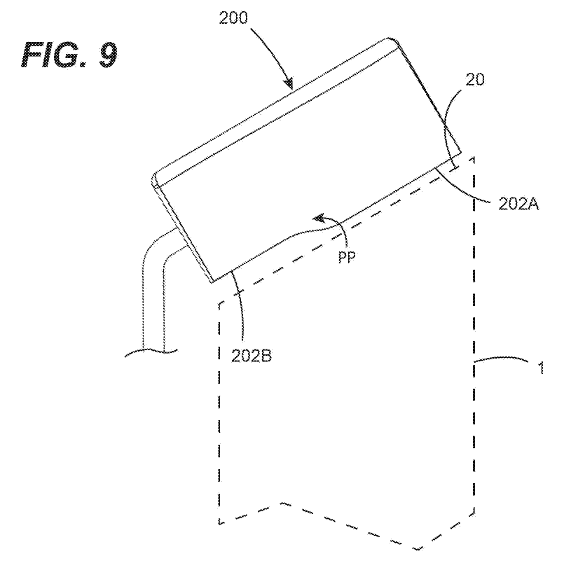

The S-shaped (and optionally stepped) configuration of wiper bodies in accordance with the disclosures made herein is believe to (i.e., not wishing to be bound to any limiting theory) enable the creation of a pivot point PP (FIG. 9) of pressure that translates to two (2) distinct “pressure pockets” or at least pressure gradients. These pressure pockets are believed to result in more effective cleaning of various contaminant material types instead of merely pushing debris along the imaging element surface. This accumulation may be a particular issue with recessed 30-degree taper imaging elements. The S-shaped (and optionally stepped) configuration of wiper bodies in accordance with the disclosures made herein is believed to further distribute and push the debris into these two pockets and down the pressure gradients to more effectively and deliberately guide debris from the imaging element surface and mitigate the potential of accumulation of debris. This in turn is believed to mitigate the potential higher surface tension of debris in the “recessed imaging element edge pockets” that reside at the transition between opposing edges of the imaging element and the support structure – i.e., the discontinuity in surfaces that defines the recess at each opposing side of the imaging element.

With respect to prior art wiper bodies designs, the S-shaped (and optionally stepped) configuration of wiper bodies in accordance with the disclosures made herein has been found by the inventors to provide more effective and reliable cleaning for liquid droplets as well as more sticky/viscous debris types (i.e., tissue residue, blood, etc.) which may otherwise amalgamate together and create a higher surface tension. This higher surface tension leads to total debris “imaging element-retaining forces” that are not easy to overcome, particularly for recessed 30-degree taper imaging elements or 30-degree taper imaging elements where contact pressure is difficult to generate due to non-uniform (recessed) or non-planar (30 degree) geometries.

Referring now to FIG. 9, engagement of the wiper body 200 with the imaging element 20 involves the upper portion 202A of the front edge portion 202 being fully compressed against the imaging element 20 to the point that the lower portion 202B becomes less compressedly engaged with the imaging element 20. The pivot point PP of the front edge portion 202 engages with the imaging element 20. In some embodiments, lower portion 202B started deflecting (i.e., deforming) before engaging the imaging element 20 as a result of the upper portion 202A engages imaging element 20 causing the front edge portion 202 to absorbs the stress along its length, thereby changing the angle of approach and pressure profile as compared to a conventional straight-edge wiper found in prior art cleaning devices.

Although disclosed herein mainly in the context of surgical scopes, wipers in accordance with embodiments of the disclosures made herein may be useful for imaging elements in a host of other applications – e.g., imaging elements in automotive applications, robotic applications, and other applications having imaging element configurations that may benefit from wipers as disclosed herein.

Although the invention has been described with reference to several exemplary embodiments, it is understood that the words that have been used are words of description and illustration, rather than words of limitation.

What is claimed is:

1. An endoscope cleaning device, comprising:
a user interface body having a laparoscope engagement portion and an elongated body engagement portion, wherein the endoscope engagement portion having engagement surfaces each matingly engageable with a respective engagement surface of a user interface portion of an endoscope for retaining the endoscope in a fixed seated position relative to a centerline longitudinal axis of the user interface body relative to the user interface body and wherein a first reference plane extends vertically through the centerline longitudinal axis of the user interface body; and
an elongated body fixedly attached at a proximate end portion thereof to the elongated body engagement portion of the user interface body, wherein the elongated body includes a first elongated passage extending along a length of the elongated body and a second elongated passage extending along the length of the elongated body, wherein the first elongated passage has an extension portion of the endoscope matingly engaged therein when the endoscope is engaged with the user interface body in the fixed seated position, wherein a longitudinal centerline axis of the first elongated passage extends parallel with a longitudinal centerline axis of the second elongated passage, wherein a second reference plane extends through both the longitudinal centerline axis of the first elongated passage and the longitudinal centerline axis of the second elongated passage, and wherein the second reference plane is rotationally offset from the first reference plane such that the first and second planes intersect along a reference axis that extends parallel to the centerline longitudinal axis of the user interface body.

2. The endoscope cleaning device of claim 1, wherein:
the elongated body is an extruded article;
the first elongated passage is a first lumen of the extruded article; and
the second elongated passage is a second lumen of the extruded article.

3. The endoscope cleaning device of claim 1, wherein the longitudinal centerline axis of the second elongated passage extends parallel to the vertically-extending plane.

4. The endoscope cleaning device of claim 3, wherein:
the elongated body is an extruded article;
the first elongated passage is a first lumen of the extruded article; and
the second elongated passage is a second lumen of the extruded article.

5. The endoscope cleaning device of claim 4, wherein the elongated body being fixedly attached at the proximate end portion thereof to the user interface body includes the elongated body being fixedly attached at the proximate end portion thereof to the user interface body to inhibit movement of the user interface body relative to the proximate end portion of the elongated body.

6. The endoscope cleaning device of claim 3, wherein the elongated body being fixedly attached at the proximate end portion thereof to the user interface body includes the elongated body being fixedly attached at the proximate end portion thereof to the user interface body to inhibit movement of the user interface body relative to the proximate end portion of the elongated body.

7. The endoscope cleaning device of claim 1, wherein the elongated body being fixedly attached at the proximate end portion thereof to the user interface body includes the elongated body being fixedly attached at the proximate end portion thereof to the user interface body to inhibit movement of the user interface body relative to the proximate end portion of the elongated body.

8. The endoscope cleaning device of claim 1, wherein the first elongated passage and the second elongated passage both extend fully to an end face of the elongated body at a distal end portion thereof.

9. The endoscope cleaning device of claim 1, wherein the vertically-extending plane bisects a lens of the endoscope when the endoscope is engaged with the user interface body in the fixed seated position.

10. An endoscope cleaning device, comprising:
a user interface body having an endoscope engagement portion and an elongated body engagement portion, wherein the endoscope engagement portion has engagement surfaces each matingly engageable with a respective engagement surface of a user interface portion of an endoscope for retaining the endoscope in a fixed seated position relative to a centerline longitudinal axis of the user interface body relative to the user interface body, and wherein a first reference plane extends vertically through the centerline longitudinal axis of the user interface body;
an elongated body fixedly attached at a proximate end portion thereof to the elongated body engagement portion of the user interface body, wherein the elongated body includes a first elongated passage extending along a length of the elongated body and a second elongated passage extending along at least a portion of the length of the elongated body, wherein a centerline longitudinal axis of the first elongated passage extends colinearly with the centerline longitudinal axis of the user interface body, and wherein the first elongated passage has an extension portion of the endoscope matingly engaged therein when the endoscope is engaged with the user interface body in the fixed seated position;
a wiper body longitudinally spaced away from a distal end face of the elongated body; and
a coupling element having a proximate end portion within the user interface body, a central portion extending within the second elongated passage, and a distal end portion extending from within the second elongated passage beyond the distal end face of the elongated body along a longitudinal centerline axis extending parallel to the centerline longitudinal axis of the first elongated passage , wherein a second reference plane extends through both the longitudinal centerline axis of the first elongated passage and the longitudinal centerline axis extending parallel to the centerline longitudinal axis of the first elongated passage, wherein the second reference plane is rotationally offset from the first reference plane such that the distal end portion of the coupling element is laterally spaced away from the first reference plane, and wherein the wiper body is fixedly attached to the distal end portion of the coupling element whereby rotational movement of the coupling element about a longitudinal axis thereof causes a corresponding rotational movement of the wiper body.

11. The endoscope cleaning device of claim 10, wherein:
the elongated body is an extruded article;
the extruded article includes a second elongated passage extending along a length of the elongated body;
the coupling element extends through at least a portion of the second elongated passage
the first elongated passage is a first lumen of the extruded article; and
the second elongated passage is a second lumen of the extruded article.

12. The endoscope cleaning device of claim 11, wherein the longitudinal centerline axis of the second elongated passage extends parallel with the vertically-extending plane.

13. The endoscope cleaning device of claim 12, wherein:
the elongated body is an extruded article;
the extruded article includes a second elongated passage extending along a length of the elongated body;
the first elongated passage is a first lumen of the extruded article; and
the second elongated passage is a second lumen of the extruded article.

14. The endoscope cleaning device of claim 13, wherein the elongated body being fixedly attached at the proximate end portion thereof to the user interface body includes the elongated body being fixedly attached at the proximate end portion thereof to the user interface body to inhibit movement of the user interface body relative to the proximate end portion of the elongated body.

15. The endoscope cleaning device of claim 12, wherein the elongated body being fixedly attached at the proximate end portion thereof to the user interface body includes the elongated body being fixedly attached at the proximate end portion thereof to the user interface body to inhibit movement of the user interface body relative to the proximate end portion of the elongated body.

16. The endoscope cleaning device of claim 10, wherein the elongated body being fixedly attached at the proximate end portion thereof to the user interface body includes the elongated body being fixedly attached at the proximate end portion thereof to the user interface body to inhibit movement of the user interface body relative to the proximate end portion of the elongated body.

17. The endoscope cleaning device of claim 10, wherein:
the elongated body includes a second elongated passage extending along a length of the elongated body; and
the first elongated passage and the second elongated passage both extend fully to the distal end face of the elongated body at a distal end portion thereof.

18. The endoscope cleaning device of claim 10, wherein:
the extruded article includes a second elongated passage extending along a length of the elongated body;
the coupling element extends through at least a portion of the second elongated passage.

19. The endoscope cleaning device of claim 10, wherein the vertically-extending plane bisects a lens of the endoscope when the endoscope is engaged with the user interface body in the fixed seated position.

20. An endoscope cleaning device, comprising:
a user interface body having an endoscope engagement portion and an elongated body engagement portion, the endoscope engagement portion having engagement surfaces each matingly engageable with a respective engagement surface of a user interface portion of an endoscope for retaining the endoscope in a fixed seated position relative to a centerline longitudinal axis of the user interface body relative to the user interface body, wherein a first reference plane extends vertically through the centerline longitudinal axis of the user interface body;
an elongated body fixedly attached at a proximate end portion thereof to the elongated body engagement portion of the user interface body, wherein the elongated body includes a first elongated passage extending along a length of the elongated body and a second elongated passage extending along at least a portion of the length of the elongated body, wherein a centerline longitudinal axis of the first elongated passage extends colinearly with the centerline longitudinal axis of the user interface body, wherein the first elongated passage has an extension portion of the endoscope matingly engaged therein when the endoscope is engaged with the user interface body in the fixed seated position, wherein a second reference plane extends through both a longitudinal centerline axis of the first elongated passage and a longitudinal centerline axis of the second elongated passage, and wherein the second reference plane is rotationally offset from the first reference plane such that the first and second planes intersect along a reference axis that extends parallel to the centerline longitudinal axis of the user interface body;
a wiper body longitudinally spaced away from a distal end face of the elongated body, wherein the wiper body includes a first arcuate wiper edge segment having a first direction of curvature and a second arcuate wiper edge segment having a second direction of curvature segment opposite the direction of curvature of the first arcuate wiper edge segment, wherein the second arcuate wiper edge segment extends contiguously from the first arcuate wiper edge segment to jointly define a front edge portion of the wiper body, and wherein an entire portion of the front edge portion extends within a single plane; and
a coupling element having a proximate end portion within the user interface body, a central portion extending within the second elongated passage, and a distal end portion extending from within the second elongated passage beyond the distal end face of the elongated body along the longitudinal centerline axis of the second elongated passage such that the distal end portion of the coupling element is laterally spaced away from the first reference plane, and wherein the wiper body is fixedly attached to the distal end portion of the coupling element whereby rotational movement of the coupling element about a longitudinal axis thereof causes a corresponding rotational movement of the wiper body.

21. The endoscope cleaning device of claim 20 wherein the front edge portion is S-shaped.

22. The endoscope cleaning device of claim 20 wherein a width of the front edge portion is uniform along an entire length of the front edge portion.

23. The endoscope cleaning device of claim 20 wherein:
a front surface of the wiper body is flat; and
the front edge portion lies along the front surface.

24. The endoscope cleaning device of claim 20 wherein:
the front edge portion is curved; and
an entire portion of the front edge portion extends within a single plane.

25. The endoscope cleaning device of claim 20 wherein:
an entire portion of the front edge portion extends within a single plane;
a width of the front edge portion is uniform along an entire length of the front edge portion;
a front surface of the wiper body is flat; and
the front edge portion lies along the front surface.

26. The endoscope cleaning device of claim 20 wherein the front edge portion includes:
a first arcuate wiper edge segment having a first direction of curvature; and
a second arcuate wiper edge segment having a second direction of curvature segment opposite the direction of curvature of the first arcuate wiper edge segment.

27. The endoscope cleaning device of claim 20, wherein:
the extruded article includes a second elongated passage extending along a length of the elongated body;
the coupling element extends through at least a portion of the second elongated passage.

28. The endoscope cleaning device of claim 27 wherein:
a front surface of the wiper body is flat; and
the front edge portion lies along the front surface.

29. The endoscope cleaning device of claim 27 wherein:
the front edge portion is curved; and
an entire portion of the front edge portion extends within a single plane.

30. The endoscope cleaning device of claim 27 wherein the front edge portion includes:
a first arcuate wiper edge segment having a first direction of curvature; and
a second arcuate wiper edge segment having a second direction of curvature segment opposite the direction of curvature of the first arcuate wiper edge segment.
